(19) United States
(12) Patent Application Publication
(10) Pub. No.: US 2010/0125175 A1
(43) Pub. Date: May 20, 2010

(54) ICON-BASED HEALTHCARE INTERFACES BASED ON HEALTH CONDITION EVENTS

(76) Inventor: Anthony Vallone, North Canton, OH (US)

Correspondence Address: Cooper Legal Group LLC, 6505 Rockside Road, Suite 330, Independence, OH, 44131, US

(21) Appl. No.: 12/274,708

(22) Filed: Nov. 20, 2008

(51) Int. Cl. A61B 5/00

(52) U.S. Cl. 600/300

(57) ABSTRACT

A health condition record of an individual is often organized in a data-centric manner, such as various forms of medical data, medical images, and text-based anecdotal information provided by caregivers and the individual. However, it may be difficult to identify significant health condition events from such representations. A health condition record organized in an event-oriented manner may facilitate assessments of the diagnosis and prognosis of the individual, the efficacy of caregivers, and the allocation of healthcare resources. The event-oriented health condition record may also be presented to caregivers and the individual by displaying health-related icons, which may facilitate the communication of health information in a language-independent manner.

### BACKGROUND

In the field of healthcare technologies, healthcare records may be compiled indicating the health state of an individual described as one or more health conditions, such as a disease or disease risk factor, a disability, an illness, etc. The information in this record may be derived from various data sources, such as complaints of the individual, medical tests, observations by various caregivers (generally including any entity involved in servicing the healthcare of the individual), etc. The healthcare record may therefore document the onset, progression, care, and prognosis of the health conditions of the individual.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

While electronic healthcare records may be usable for evaluating the health condition of an individual according to empirical data relating to such health conditions, it may be comparatively difficult to extrapolate other information from such healthcare records. For example, the healthcare record may feature information describing the status of the individual's health condition (e.g., “individual complains of complications from diabetes; blood glucose measured at 220 mg/dL; insulin administered.”) However, information about particular events relating to the health conditions of the individual may be more difficult to extrapolate from the data captured in the healthcare record. For example, the individual may report to a caregiver a series of events related to the individual's health condition, such as incidents of high blood sugar; however, the caregiver may be more directly focused on diagnosing and treating the diabetic health condition rather than documenting the particular events reported by the individual. Thus, the healthcare record of the individual may document the events relating to the health conditions of the individual only tangentially or not at all (e.g., “individual diagnosed with diabetes.”)

It may be advantageous to orient the healthcare record of the individual to facilitate the capturing and documenting of the health condition events relating to the health conditions of the individual. This information may be of direct value in the healthcare of the individual (e.g., the accuracy and specificity of diagnostic and prognostic conclusions of a health condition may be improved by including the particular health condition events, such as the frequency and distribution of hyperglycemic and/or hypoglycemic events.) This information may also be of value in other aspects of healthcare, such as for insurance and billing purposes. For example, a healthcare organization (such as the U.S. Department of Health and Human Services) may allocate healthcare resources to the caregivers of an individual based on the frequency and distribution of health condition events related to the health conditions of the individual.

Additional advantages may be achieved by exchanging such health condition event information among caregivers through an easily understandable mode of communication. It may be appreciated that the information about the health state of the individual may be generated and shared among many caregivers (including the individual), and that an accurate conveyance of such information may be complicated by differences in the regions, cultures, languages, literacy rates, education levels, and intellectual capacities of such caregivers. It may be desirable to orient the communication of health condition event information among the caregivers for the individual (including the individual) through the use of a set of icons of predominantly pictorial form. For example, a standardized icon set may be developed for communicating information about individuals' health conditions and events related thereto, and may be incorporated in various information technologies (such as systems, devices, and user interfaces exposed therethrough.) Such icons may be more readily, widely, and consistently understandable as compared with other modes of communication, such as text, numeric data, and charts and other medical illustrations.

Accordingly, healthcare information technology systems, devices, and user interfaces may be devised that record a health condition of an individual according to the health condition events significantly related thereto (e.g., by documenting the diagnosis and prognosis of a diabetic condition of an individual according to incidents of hyperglycemia and hypoglycemia.) This captured information may then be communicated among the caregivers for the individual (including the individual) through the use of healthcare-related icons of predominantly pictorial form. Healthcare-related information technology systems configured in this manner may therefore facilitate the provision of healthcare to the individual in both clinical aspects (e.g., by improving the convenience, speed, and accuracy of communicated information among such caregivers) and non-clinical aspects (e.g., by permitting a more efficient allocation of healthcare resources by a healthcare agency according to the needs of the individual, as illustrated by the recorded health condition events of the health conditions of the individual.)

To the accomplishment of the foregoing and related ends, the following description and annexed drawings set forth certain illustrative aspects and implementations. These are indicative of but a few of the various ways in which one or more aspects may be employed. Other aspects, advantages, and novel features of the disclosure will become apparent from the following detailed description when considered in conjunction with the annexed drawings.

### DESCRIPTION OF THE DRAWINGS

FIG. 1 is a Uniform Markup Language (UML) diagram illustrating an exemplary representation of a health condition record.

FIG. 2 is an exemplary health condition record represented according to the exemplary representation of FIG. 1.

FIG. 3 is a flow chart illustrating an exemplary computer-implemented method of representing a health condition of an individual.

FIG. 4 is a component block diagram illustrating an exemplary system for representing a health condition of an individual.

FIG. 5 is a flow chart illustrating another exemplary computer-implemented method of representing a health condition of an individual.

FIG. 6 is an illustration of an exemplary automated diagnosis of a health condition of an individual based on a set of health condition circumstance descriptors.

FIG. 7 is an illustration of an exemplary medication reminder device configured to display health-related icons representing health condition action descriptors associated with a health condition event descriptor in an event-oriented health condition record.

FIG. 8 illustrates an exemplary computing environment wherein one or more of the provisions set forth herein may be implemented.

### DETAILED DESCRIPTION

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

Information technology systems in the field of healthcare often involve a representation of a health state of an individual. The health state is often described and represented in terms of one or more health conditions, such as a disease, illness, injury, or disability. The health condition, in turn, may be represented through a variety of recorded data, such as descriptions of the health condition or sensory perceptions by the individual, results of medical tests, clinical observations of caregivers, and records of remedial actions performed to address the health condition or its symptoms. This set of data may provide a depiction of the cause, onset, progression, and prognosis of the health conditions of the individual.

Healthcare information technology systems may also be configured to facilitate the receiving of this data from the caregivers (including the individual), the analysis of such data by caregivers and automated processes, the communication of such data to caregivers (including the individual), and the automated exchange of such information with other devices. These tasks are often oriented around representations of the health conditions as a set of qualitative and/or quantitative measurements of the status of the health condition, which may be recorded and represented in absolute terms or relative to other measurements of the health condition of the individual, exemplary cases, medical standards, etc. A caregiver may find a measurement-oriented description of this nature useful in the assessment and treatment of the health condition. For example, a caregiver tasked with assessing and treating a diabetic condition of an individual may record periodic assessments of the individual, such as blood glucose levels, and the remedial measures undertaken to address the evaluated health state of the individual (e.g., the prescription of diabetic control medications.) However, the caregiver may fail to record episodic events, such as the incidents of hyperglycemia or hypoglycemia reported by the individual during a particular period, because such event-based information may be of limited value to the caregiver in the immediate task of evaluating and treating the diabetic health condition.

However, healthcare data may be used in many types of analysis and representation, and for some forms of analysis and representation, a measurement-oriented representation of the health condition of an individual may be less helpful than other representations. As a first example, a second caregiver who is tasked with treating the diabetic health condition of the individual may wish to review the events associated with the diabetic health condition, such as onset, frequency, and distribution of hyperglycemic and hypoglycemic conditions. As a second example, a researcher may wish to review event-related information for a particular health condition for a particular individual, for a group of individuals (e.g., an epidemiologist reviewing a set of healthcare records to determine patterns of events among victims of a particular disease.) As a third example, an insurance company may wish to evaluate the health risks of an individual based on an assessment of the episodic history of the health condition of the individual, or based on event-related information of other individuals with the health condition. As a fourth example, a healthcare organization, such as a public health agency, may wish to utilize event-related information in order to allocate healthcare resources in an efficient manner (e.g., by compensating caregivers and assessing performance based on the health condition of a treated individual, as evidenced by the health condition events experienced by the individual before, during, and after treatment.) However, information about the events associated with health conditions of one or more individuals may be only partially documented by first-line caregivers dealing with the immediate task of assessing and treating the health conditions of the individuals. This may be particularly true if the healthcare record of the individual is predominantly focused on measurement and observation data, and is not oriented to record health condition events as a significant aspect of the health condition.

In addition, the communication of health condition information about the health conditions of an individual among caregivers (including the individual) may be complicated by various factors, such as regional and language differences and variations in educational backgrounds, literacy, and intellectual capacities. For example, a representation of a health condition recorded by a first caregiver may be reviewed by a second caregiver in another country who speaks a different language; by peripheral caregivers, such as pharmacists and home caregivers who assist the individual in a home setting; by an academic researcher reviewing the health condition record in a research context; by agents of health agencies, such as a department of public health or an adjuster in an insurance company; and by family members of the individual. Each of these individuals may have difficulty understanding the contents of the healthcare record of the individual if expressed in text, numeric data (such as results of medical tests), charts, and medical images. A more advantageous presentation of this information to caregivers (including the individual) involves the use of icons of predominantly pictorial form that represent both the health condition and the health condition event. The use of icons for representing such information may have several advantages. As a first example, caregivers may comprehend the health condition and health condition event information in a language-independent manner. Pictographic icons can be used to circumvent differences in language capabilities and skill levels among the caregivers and the individual. The icon set may be cognizable to individuals with limited literacy skills or with mental faculties, such as very young, elderly, and mentally handicapped individuals. Second, if a common set of medical icons is utilized by many caregivers and individuals, it may become a standard and standardized lexicon for the communication of health-related information, including medication regimen information. Regular and frequent exposure to the same icon set, especially from a variety of sources, may lead to quicker, fuller, and more accurate recognition and comprehension by caregivers and individuals of these icons and the illustrated concepts. Moreover, widespread exposure to and use of the same icon set may improve the speed, depth, and precision of communication of health-related information among many caregivers and individuals.

In view of these scenarios associated with the representation of health condition information, techniques may be devised for representing the health condition of an individual that ameliorate some of these difficulties and facilitate some of these uses. These techniques involve representing the health condition of the individual as a health condition descriptor associated with a set of health condition event descriptors, each representing an event associated with the health condition (e.g., a representation of a diabetic health condition of an individual as a set of hyperglycemic or hypoglycemic events.) If the healthcare record of the individual is structured in this manner, caregivers may be persuaded to record episodic information as part of the healthcare record, thereby preserving this information for a wide variety of uses. Moreover, the information may be communicated among caregivers (including the individual) by representing both the health condition and the health condition events through the use of icons of predominantly pictorial form.

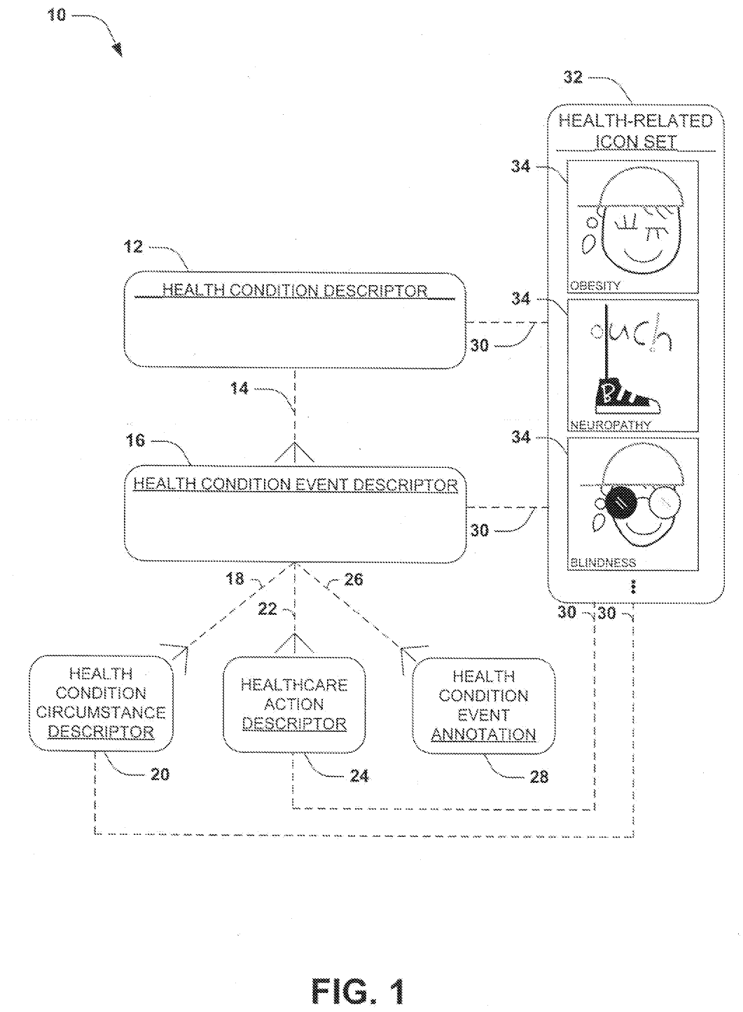

FIG. 1 presents an exemplary representation 10 of a health condition record, illustrated as a Uniform Markup Language (UML) diagram. The exemplary representation 10 includes a health condition descriptor 12, which may be selected from a set of health condition descriptors 12 representing various health conditions (e.g., a diabetes health condition descriptor, a cardiovascular disease health condition descriptor, and an epilepsy health condition descriptor.) A subset of health condition descriptors 12 may be selected form the set of health condition descriptors set to represent the health conditions afflicting an individual. Respective health condition descriptors 12 may have a one-to-many association 14 with a selected set of health condition event descriptors 16 that represent respective health condition events associated with the respectively represented health conditions. For example, a diabetes health condition descriptor 12 may be associated with three health condition event descriptors 16 representing hypoglycemia events, thereby describing a diabetic health condition of an individual according to three hypoglycemic episodes. In some embodiments, respective health condition event descriptors 16 may also be associated with other descriptors to add representations of details of the health condition events. For example, a health condition event descriptor 16 may have (e.g.) a one-to-many relationship 18 with a set of health condition circumstance descriptors 20 (which may add structured details to the representation of the represented health condition event, such as a date or time of the event, a duration, a location, or a qualitative or quantitative measurement of significance); a one-to-many relationship 22 with a set of healthcare action descriptors 24 (which may add data items representing healthcare-related actions undertaken by caregivers, including the individual, in response to the represented health condition event); and a one-to-many relationship 26 with a set of health condition event annotations 28 (which may add detailed comments to the represented health condition event, such as text-based narratives.) It may be appreciated that while (in some embodiments) these additional descriptors and details may be associated with a health condition event descriptor 16, the healthcare record of a represented individual is more predominantly oriented around the representation of a health condition according to one or more represented health condition events, in accordance with the techniques discussed herein and the advantages that may be derived therefrom.

As further illustrated in FIG. 1, various descriptors may be represented through the use of icons 34 of predominantly pictorial form, again in accordance with the techniques discussed herein. For example, respective health condition descriptors 12 and health condition event descriptors 16 (as well as health condition circumstance descriptors 20 and health action descriptors 22) may have a one-to-one relationship 30 with respective icons 34 in a health-related icon set 32, and a health condition of an individual may be presented for a caregiver (including the individual) by retrieving and displaying the associated icons 34 of the health-related icon set 32. The presentation of the health condition record in this manner may facilitate the rapid, consistent, and language-independent communication of health-related information of a represented individual among caregivers.

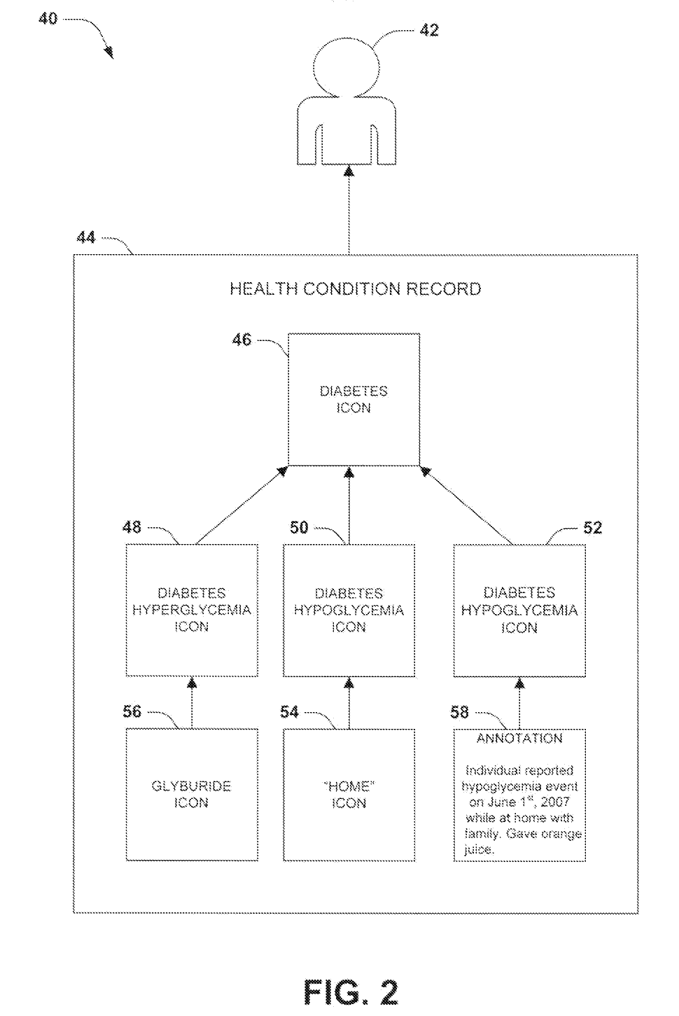

In accordance with the exemplary representation 10 of health condition records illustrated in FIG. 1, FIG. 2 presents an exemplary scenario 4 featuring an exemplary health condition record 44 representing a diabetic health condition of a particular individual 42. The health condition record 44 may comprise a health condition descriptor 46, such as a “diabetes” health condition descriptor indicating that the individual 42 is afflicted with diabetes. The health condition descriptor 46 may be associated with a set of health condition event descriptors that represent health condition events relating to the represented health condition. For example, the health condition descriptor 46 in the exemplary health condition record 44 of FIG. 2 is associated with a first health condition event descriptor 48 representing a hyperglycemic event, a second health condition event descriptor 50 representing a first hypoglycemic event, and a third health condition event descriptor 52 representing a second hypoglycemic event. Together, these health condition event descriptors describe the episodic history of the diabetes condition of the individual 42. As further indicated in the exemplary representation 10 of FIG. 1, in some embodiments, additional data items may be associated with the health condition event descriptors. For example, in the exemplary health condition record 44 of FIG. 2, additional data items are associated with the health condition event descriptors; e.g., a health condition circumstance descriptor 54 is associated with the second health condition event descriptor 50 indicating that the event occurred at the home of the individual 42; a healthcare action descriptor 56 is associated with the first health condition event descriptor 50 indicating that the individual 42 was prescribed a diabetes medication (“Glyburide”) in response to the first hyperglycemic event; and a health condition event annotation 58 is associated with the third health condition event descriptor 52 that provides a clinical narrative of the second hypoglycemic event and responsive treatment of the individual 42 (indicating that a serving of orange juice was given to the individual to address the second hypoglycemic event.) Finally, as indicated in FIG. 1, the descriptors of FIG. 2 are represented by displaying icons of predominantly pictorial form, which may communicate the health-related information more quickly and conveniently to other caregivers (including the individual) than may be achieved through the presentation of text, data, and/or medical illustrations.

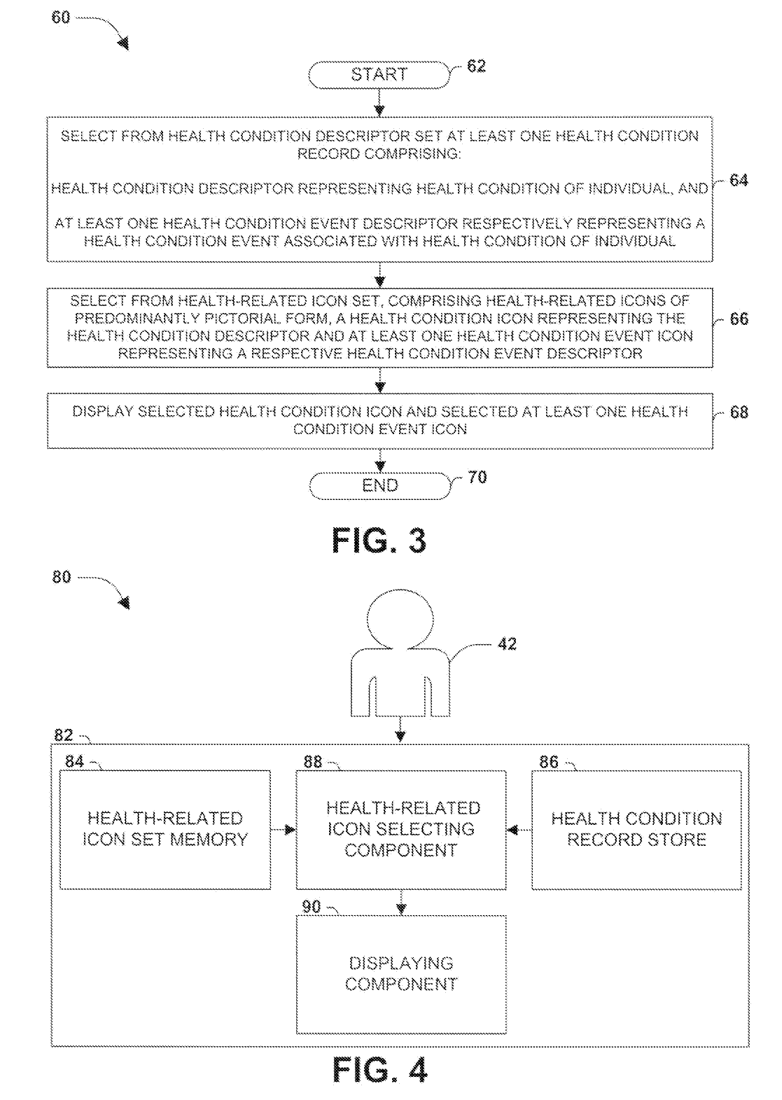

FIG. 3 presented a first embodiment 60 of the techniques presented herein, illustrated as an exemplary computer-implemented method 60 of representing a health condition of an individual. The exemplary computer-implemented method 60 begins at 62 and involves selecting 64 from a health condition descriptor set at least one health condition record, wherein the health condition record comprises a health condition descriptor representing the health condition of the individual, and at least one health condition event descriptor respectively representing a health condition event associated with the health condition of the individual. The exemplary computer-implemented method 60 also involves selecting 66 from a health-related icon set, comprising health-related icons of predominantly pictorial form, a health condition icon representing the health condition descriptor and at least one health condition event icon representing a respective health condition event descriptor. The exemplary computer-implemented method 60 also involves displaying 68 the selected health condition icon and the selected at least one health condition event icon. Having generated and presented a health condition representation of the health condition record the use of icons of predominantly pictorial form, the exemplary computer-implemented method 60 thereby achieves the language-independent and event-oriented representation of the health condition(s) of the individual, and so ends at 70.

FIG. 4 presents a second embodiment of the techniques discussed herein, illustrated as an exemplary scenario 80 featuring an exemplary system 82 for representing a health condition of an individual 42. The exemplary system 82 comprises a health-related icon set memory 84, which is configured to store a health-related icon set 32 comprising health-related icons 34 of predominantly pictorial form. The exemplary system 82 also comprises a health condition record store 86, which is configured to store at least one health condition record comprising a health condition descriptor representing the health condition of the individual 42, and at least one health condition event descriptor respectively representing a health condition event associated with the health condition of the individual 42. The exemplary system 82 also includes a health-related icon selecting component 88, which is configured to select from the health-related icon set memory 84 a health condition icon representing the health condition descriptor and at least one health condition event icon representing a respective health condition event descriptor. Finally, the exemplary system 82 includes a displaying component 90, which is configured to display the selected health condition icon and the selected at least one health condition event icon. Having achieved the displaying of icons in order to present an event-based representation of the health conditions of the individual 42, the exemplary system 82 achieves the communicating of the health condition record of the individual 42 in a more convenient and consistent manner than other representations.

In view of the exemplary scenarios of FIGS. 1-2 and the exemplary embodiments illustrated in FIGS. 3-4, it may be appreciated that the displaying of event-oriented health condition records using health-related icons is rendered from a health condition record 44 of the individual 42. The health condition record 44 may be generated in many ways, such as an automated process that generates health condition records 44 based on various data sources, or that transforms existing data sources into event-oriented health condition records 44. Alternatively or additionally, the same icons that are useful for displaying the information in a health condition record 44 of an individual 42 may be used to facilitate a caregiver in preparing the health condition record 44 of the individual 42, such as in an icon-based data entry system.

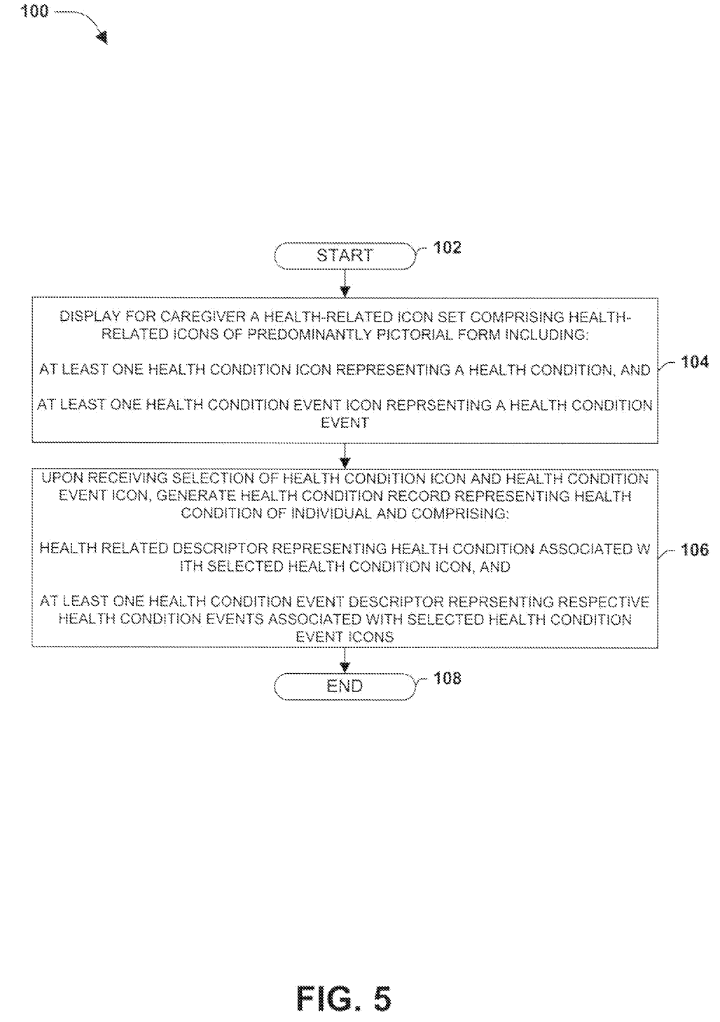

FIG. 5 illustrates an exemplary embodiment of these techniques, comprising an exemplary computer-implemented method 100 of representing a health condition of an individual. The exemplary computer-implemented method 100 begins at 102 and involves displaying 104 for a caregiver a health-related icon set comprising health-related icons of predominantly pictorial form, including at least one health condition icon representing a health condition and at least one health condition event icon representing a health condition event related to a health condition. The exemplary computer-implemented method 100 also involves, upon receiving a selection of a health condition icon representing a health condition of the individual and at least one health condition event icon representing at least one health condition event related to the selected health condition, generating 106 a health condition record representing the health condition of the individual, where the health condition record comprises a health condition descriptor representing the health condition associated with the selected health condition icon and at least one health condition event descriptor representing respective health condition events associated with the selected health condition event icons. Having generated the event-oriented health condition record through the displaying of health-related icons in a user interface, the exemplary computer-implemented method 100 thereby facilitates a caregiver in generating the health condition record of the individual, and so ends at 108.

The techniques discussed herein may be devised with variations in many aspects, and some variations may present additional advantages and/or reduce disadvantages with respect to other variations of these and other techniques. Moreover, some variations may be implemented in combination, and some combinations may feature additional advantages and/or reduced disadvantages through synergistic cooperation. The variations may be incorporated in various embodiments (e.g., the exemplary method 60 of FIG. 3 and the exemplary system 82 of FIG. 4) to confer individual and/or synergistic advantages upon such embodiments.

A first aspect that may vary among embodiments of these techniques relates to the types of information stored in the health condition record 44. It may be appreciated that while the health condition record 44 includes at least one health condition descriptor 12 associated with at least one health condition event descriptor 16, other types of information may also be included. FIGS. 1 and 2 illustrate some examples of such supplemental information, which may be presented (e.g.) through the displaying of icons of predominantly pictorial form that are stored in the health-related icon set 32 and associated with the descriptors representing the health condition(s) and health condition event(s).

As a first example, at least one health condition event descriptor 16 of the health condition record 44 may be associated with at least one health condition circumstance descriptor 20, which may representing at least one health circumstance of the associated health condition event. For example, various health condition circumstance descriptors 20 may add information describing the event, such as the date or duration of the event, the location of the event, a qualitative and/or quantitative degree of the event, and a cause or symptom of the event. Multiple health condition circumstance descriptors 20 may also be added to supplement the health condition event descriptor 16 with many pieces of information. Moreover, the health condition circumstance descriptors 20 may be associated with health-related icons, which may be selected and displayed along with the icons associated with the health condition descriptor 12 and health condition event descriptor 16.

As a second example of this first aspect, the health condition record 44 may also associate a health condition event descriptor 16 with one or more health condition action descriptors 24 that describe actions taken by caregivers in response to the health condition event. Such actions may include (e.g.) healthcare tests performed on the individual (such as diagnostic or prognostic tests, or eligibility or efficacy tests of potential remedial actions); healthcare procedures, such as medical or surgical intervention; and/or the prescription of one or more medications or services (such as home healthcare services) in response to the event. The health condition action descriptors 24 may also be associated with health-related icons, which may be selected and displayed along with the icons associated with the health condition descriptor 12 and health condition event descriptor 16.

As a third example of this first aspect, the health condition record 44 may also associate a health condition annotation 28 with a health condition event descriptor 16. Whereas other types of descriptors (such as health condition circumstance descriptors 20 and health condition action descriptors 24) may supplement a represented health condition event with well-structured details, a health condition annotation 28 may enable a caregiver to supplement the represented health condition event with less structured details, such as a textual or recorded narrative by a caregiver (including the individual) or device-generated data, such as medical images or charts. The health condition annotation(s) 28 associated with a health condition event descriptor 16 may be displayed, played, or otherwise rendered along with the icons representing the descriptors representing the health condition and health condition event(s). Those of ordinary skill in the art may devise many types of information that may be added to the health condition record 44 of the individual 42 in accordance with the techniques discussed herein.

A second aspect that may vary among embodiments of these techniques involves other elements that may be included in such embodiments to provide additional advantages and/or to reduce disadvantages. For example, additional elements may be added to the exemplary computer-implemented methods of FIGS. 3 and/or 5, and additional components may be added to the exemplary system 82 of FIG. 4, to extend the functionalities and capabilities of such systems and methods.

As a first example of this second aspect, an embodiment may both display the event-oriented health condition record and facilitate its entry through the displaying of health-related icons in a user interface. For example, an exemplary system may include a displaying component that is configured to display the health condition icons and health condition event icons for a caregiver, and a health record compiling component configured to generate the health condition record based on data entry (e.g., the selection of icons) from the caregiver, which may be received through an input device such as a keyboard or mouse. As one example, the health record compiling component may be configured to receive from the caregiver a selected health condition icon representing a health condition of the individual and at least one selected health condition event icon representing at least one health condition event associated with the selected health condition of the individual. The health record compiling component may then be configured to compile a selected health condition descriptor represented by the selected health condition icon and at least one health condition event descriptor respectively represented by the selected at least one health condition event icon. Alternatively or additionally, embodiments may also facilitate the entry by caregivers of health condition event annotations. For example, a system embodiment (such as the exemplary system 82 of FIG. 4) may include a health condition event annotating component, which may be configured to receive from a caregiver at least one health condition event annotation associated with at least one health condition event of the individual and store the at least one health condition event annotation in the health condition record of the individual stored in the health condition record memory; and the displaying component 90 may be configured to display the at least one health condition event annotation with the at least one health condition event icon of the associated at least one health condition event. In this manner, embodiments of these techniques may facilitate both the generating and the displaying of the health condition record of an individual.

Alternatively or additionally, the health condition record may be generated, in whole or in part, based on automated processes, such as an automated compiling of a health condition record based on data detected by health-related detectors. Accordingly, in some embodiments of these techniques, at least one health condition circumstance descriptor may be detected by at least one detection component, which may be configured to detect a measurement of the health condition of the individual, select a health condition circumstance descriptor that describes the measurement from the health condition descriptor set; and store the selected health condition circumstance descriptor in the health condition record. For example, a blood glucose meter configured to detect hyperglycemic and/or hypoglycemic events associated with a diabetic health condition. The compiled health condition record is represented in an event-oriented manner and may be presented to caregivers (including the individual) through the displaying of health-related icons of predominantly pictorial form that are associated with the automatically compiled health condition record. However, those of ordinary skill in the art may devise many ways of compiling the health condition record in accordance with the techniques discussed herein.

As a second example of this second aspect, in addition to representing the health condition of the individual in an event-oriented manner and presenting it to caregivers (including the individual) by displaying health-related icons, embodiments of these techniques may also include a diagnostic capability that selects a health condition of the individual as a health condition diagnosis. The health condition events and associated health condition event descriptors may be evaluated to determine a likely health condition diagnosis, which may be included in the health condition record and/or represented for caregivers, such as through the displaying of health-related icons. For example, embodiments of these techniques may involve generating at least one health condition diagnosis representing the at least one health condition of the individual based on the at least one health condition event using a diagnostic database comprising at least one correlationally weighted relationship between respective health condition events and respective health condition diagnoses. The diagnostic database may be developed, e.g., by healthcare professionals who may attribute diagnostic weights to various health condition diagnoses (and health condition descriptors representing such diagnoses), through automated evaluation of historic data to identify correlations between symptoms and diagnoses, etc. Such embodiments may also involve selecting the health-related icons comprising: selecting from the health-related icon set the at least one health condition icon representing the at least one health condition represented by the at least one health condition diagnosis.

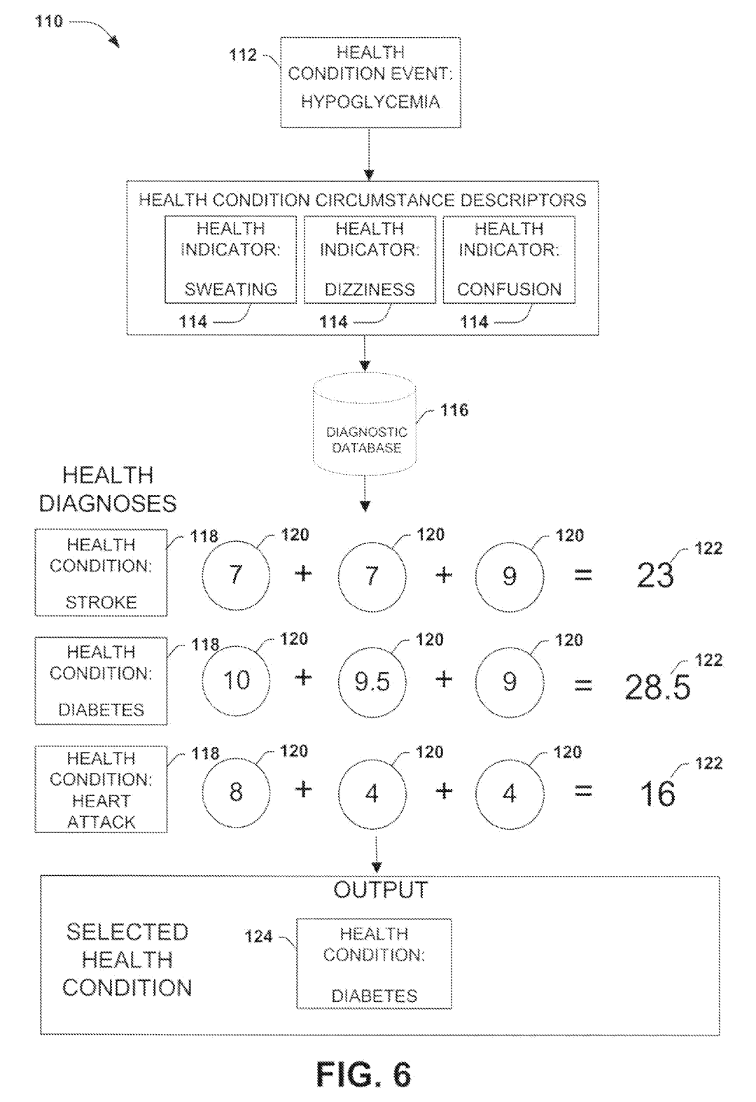

FIG. 6 presents one such embodiment, illustrated as an exemplary scenario 110 involving an automated diagnosis of a health condition and a selection of a health condition descriptor associated with the diagnosis using a diagnostic database 116. In this embodiment, the generating of a diagnosis begins with a health condition event descriptor 112 (such as an incident of hypoglycemia) and a set of health condition circumstance descriptors 114 associated with the health condition event descriptor 112. These health condition circumstance descriptors 114 may be compared with a diagnostic database 116 that attributes correlational diagnostic weights between various health condition circumstance descriptors 114 and various health condition diagnoses 118. Accordingly, diagnostic weights 120 may be selected from the diagnostic database 116 for the health condition circumstance descriptors 116 for respective health condition diagnoses 118. The automated process may then compute an aggregate diagnostic weight 120 for respective health condition diagnoses 118 based on the selected diagnostic weights 120 of the health condition circumstance descriptors 114 for the health condition diagnoses 118, and selecting at least one health condition diagnosis 124 having a significant aggregate diagnostic weight 122. Additional details and examples are presented in U.S. patent application Ser. No. 11/858,764, entitled “Automated Correlational Health Diagnosis,” which (excepting the claims) is incorporated herein by reference. Those of ordinary skill in the art may devise many techniques for generating health condition diagnoses based on the information in a health condition record while implementing the techniques discussed herein.

A third aspect that may vary among embodiments of these techniques relates to the displaying of health-related icons in order to present the health condition record of the individual to caregivers (including the individual) in an easily understandable manner. As a first example, the health-related icons may be displayed in many ways, such as a timeline layout that representing a chronological health history of the health condition of the individual (e.g., by organizing the visual layout of the icons by date), and/or a descriptive layout representing a descriptive health history of the health condition of the individual (e.g., by organizing the visual layout of the icons according to health conditions, and/or the significance of various health condition events to the health state of the individual.)

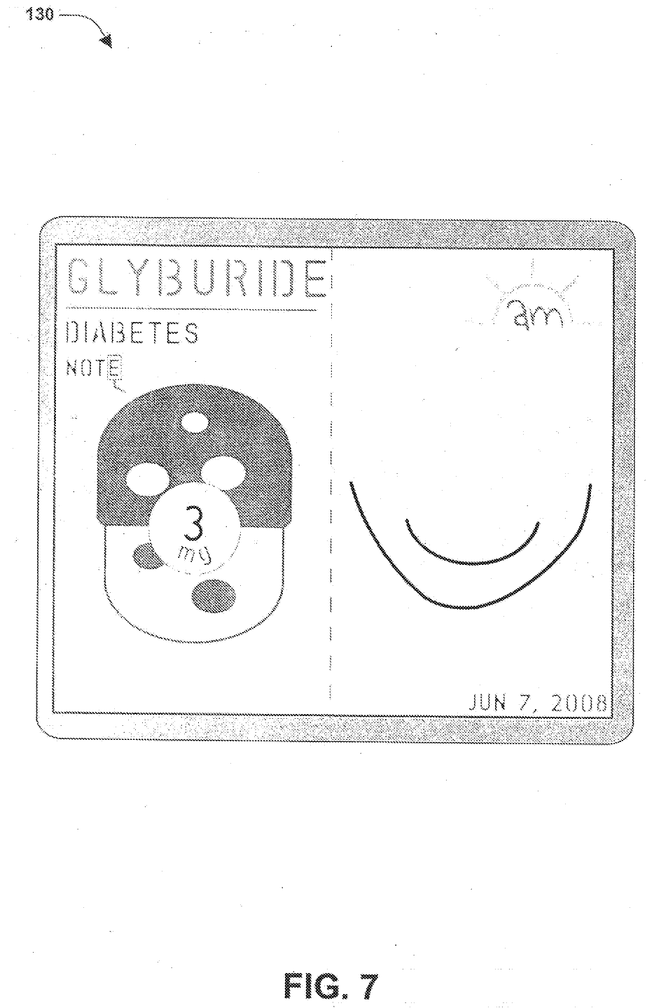

As a second example, the health-related icons representing the health condition(s) of the individual may be displayed on various devices, which may assist in various aspects of the healthcare of the individual. FIG. 7 illustrates one such embodiment, where at least one healthcare action descriptor represents at least one healthcare action comprising at least one medication prescribed for the individual associated with at least one health condition of the individual. This medication prescription may be represented to the individual by displaying the corresponding health-related icon on a medication device 130, which may be presented to the individual as a reminder for taking a prescribed medication at a designated time (e.g., for taking a dose of a diabetic medication named “Glyburide” for control of blood sugar.) For example, the medication device 130 may be configured to store the at least one medication instruction and generate a medication reminder message by displaying icons of predominantly pictorial form from a medication regimen icon set that pertain to the medication instruction. Additional details and examples are presented in U.S. patent application Ser. No. 11/712,376 (“Device for Facilitating Compliance With Medication Regimen”), which (excepting the claims) is incorporated herein by reference.

A second device that may be used to display health-related icons relating to an event-oriented health condition record of an individual may be utilized where at least one healthcare action descriptor represents at least one healthcare action comprising at least one healthcare service prescribed for the individual associated with at least one health condition of the individual. In order to facilitate the performance of such healthcare services for the individual, a service task facilitator device may be provided to a caregiver. For example, the health-related icons may be displayed by programming at least one service task instruction associated with the prescribed healthcare service into a service task facilitator device configured to store the at least one service task instruction and display service task icons representing the service task instructions. This device may therefore be used by the caregiver during the performance of the healthcare services for the individual. Additional detail and examples are presented in U.S. Patent Application No. 61/018,191 (“Icon-Based Facilitation of Service Task Performance”), which (excepting the claims) is incorporated herein by reference. Those of ordinary skill in the art may devise many ways of displaying the health-related icons of event-oriented health condition record, such as in various layouts and through various devices, according to the techniques discussed herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this application, the terms “component,” “module,” “system”, “interface”, and the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, those skilled in the art will recognize many modifications may be made to this configuration without departing from the scope or spirit of the claimed subject matter.

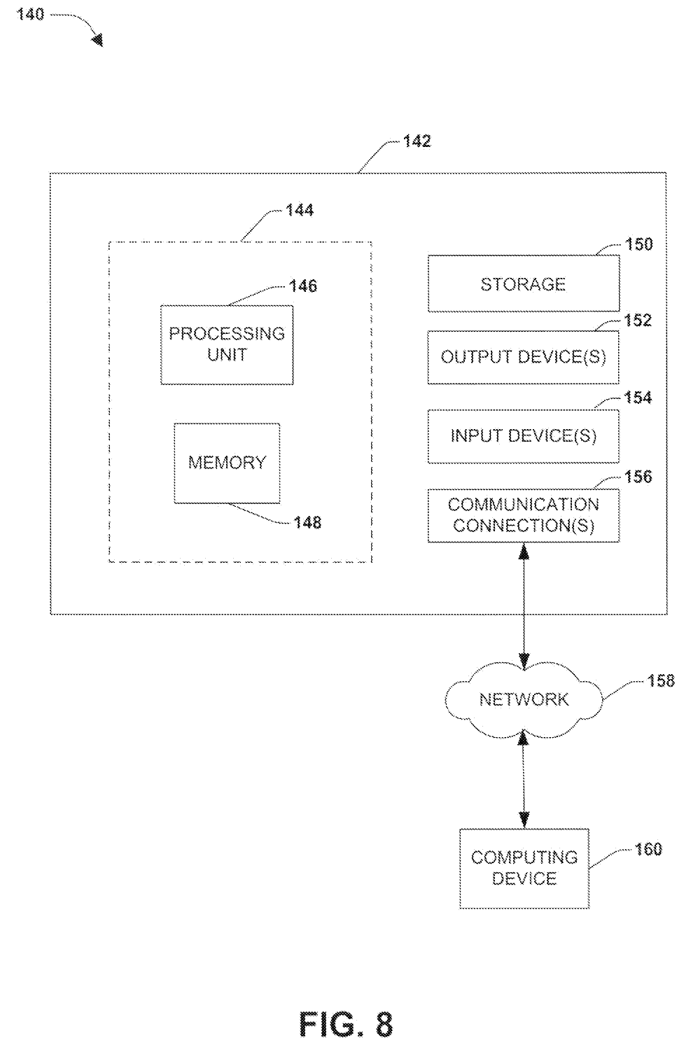

FIG. 8 and the following discussion provide a brief, general description of a suitable computing environment to implement embodiments of one or more of the provisions set forth herein. The operating environment of FIG. 8 is only one example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, Personal Digital Assistants (PDAs), media players, and the like), multiprocessor systems, consumer electronics, mini computers, mainframe computers, distributed computing environments that include any of the above systems or devices, and the like.

Although not required, embodiments are described in the general context of “computer readable instructions” being executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

FIG. 8 illustrates an example of a system 140 comprising a computing device 142 configured to implement one or more embodiments provided herein. In one configuration, computing device 142 includes at least one processing unit 146 and memory 148. Depending on the exact configuration and type of computing device, memory 148 may be volatile (such as RAM, for example), non-volatile (such as ROM, flash memory, etc., for example) or some combination of the two. This configuration is illustrated in FIG. 8 by dashed line 144.

In other embodiments, device 142 may include additional features and/or functionality. For example, device 142 may also include additional storage (e.g., removable and/or non-removable) including, but not limited to, magnetic storage, optical storage, and the like. Such additional storage is illustrated in FIG. 8 by storage 150. In one embodiment, computer readable instructions to implement one or more embodiments provided herein may be in storage 150. Storage 150 may also store other computer readable instructions to implement an operating system, an application program, and the like. Computer readable instructions may be loaded in memory 148 for execution by processing unit 146, for example.

The term “computer readable media” as used herein includes computer storage media. Computer storage media includes volatile and nonvolatile, removable and non-removable media implemented in any method or technology for storage of information such as computer readable instructions or other data. Memory 148 and storage 150 are examples of computer storage media. Computer storage media includes, but is not limited to, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which can be used to store the desired information and which can be accessed by device 142. Any such computer storage media may be part of device 142.

Device 142 may also include communication connection(s) 156 that allows device 142 to communicate with other devices. Communication connection(s) 156 may include, but is not limited to, a modem, a Network Interface Card (NIC), an integrated network interface, a radio frequency transmitter/receiver, an infrared port, a USB connection, or other interfaces for connecting computing device 142 to other computing devices. Communication connection(s) 156 may include a wired connection or a wireless connection. Communication connection(s) 156 may transmit and/or receive communication media.

The term “computer readable media” may include communication media. Communication media typically embodies computer readable instructions or other data in a “modulated data signal” such as a carrier wave or other transport mechanism and includes any information delivery media. The term “modulated data signal” may include a signal that has one or more of its characteristics set or changed in such a manner as to encode information in the signal.

Device 142 may include input device(s) 154 such as keyboard, mouse, pen, voice input device, touch input device, infrared cameras, video input devices, and/or any other input device. Output device(s) 152 such as one or more displays, speakers, printers, and/or any other output device may also be included in device 142. Input device(s) 154 and output device(s) 152 may be connected to device 142 via a wired connection, wireless connection, or any combination thereof. In one embodiment, an input device or an output device from another computing device may be used as input device(s) 154 or output device(s) 152 for computing device 142.

Components of computing device 142 may be connected by various interconnects, such as a bus. Such interconnects may include a Peripheral Component Interconnect (PCI), such as PCI Express, a Universal Serial Bus (USB), firewire (IEEE 1394), an optical bus structure, and the like. In another embodiment, components of computing device 142 may be interconnected by a network. For example, memory 148 may be comprised of multiple physical memory units located in different physical locations interconnected by a network.

Those skilled in the art will realize that storage devices utilized to store computer readable instructions may be distributed across a network. For example, a computing device 160 accessible via network 158 may store computer readable instructions to implement one or more embodiments provided herein. Computing device 142 may access computing device 160 and download a part or all of the computer readable instructions for execution. Alternatively, computing device 142 may download pieces of the computer readable instructions, as needed, or some instructions may be executed at computing device 142 and some at computing device 160.

Various operations of embodiments are provided herein. In one embodiment, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein.

Moreover, the word “exemplary” is used herein to mean serving as an example, instance, or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims may generally be construed to mean “one or more” unless specified otherwise or clear from context to be directed to a singular form.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes”, “having”, “has”, “with”, or variants thereof are used in either the detailed description or the claims, such terms are intended to be inclusive in a manner similar to the term “comprising.”

What is claimed is:

1. A computer-implemented method of representing a health condition of an individual, comprising:
selecting from a health condition descriptor set at least one health condition record comprising:
a health condition descriptor representing the health condition of the individual, and
at least one health condition event descriptor respectively representing a health condition event associated with the health condition of the individual;
selecting from a health-related icon set, comprising health-related icons of predominantly pictorial form, a health condition icon representing the health condition descriptor and at least one health condition event icon representing a respective health condition event descriptor; and
displaying the selected health condition icon and the selected at least one health condition event icon.

2. The method of claim 1:
at least one health condition event descriptor of the health condition record associated with at least one health condition circumstance descriptor representing at least one health circumstance of the associated health condition event, comprising at least one of:
a date of the health condition event,
a location of the health condition event,
a degree of the health condition event,
a duration of the health condition event,
a cause of the health condition event, and
a symptom of the health condition event;
selecting the health-related icons comprising: selecting from the health-related icon set at least one health condition circumstance icon representing the at least one health condition circumstance descriptor; and
the displaying comprising: displaying the selected health condition circumstance icon.

3. The method of claim 2, at least one health condition circumstance descriptor detected by at least one detection component configured to:
detect a measurement of the health condition of the individual;
select a health condition circumstance descriptor that describes the measurement from the health condition descriptor set; and
store the selected health condition circumstance descriptor in the health condition record.

4. The method of claim 1:
the method comprising: generating at least one health condition diagnosis representing the at least one health condition of the individual based on the at least one health condition event using a diagnostic database comprising at least one correlationally weighted relationship between respective health condition circumstance descriptors associated with respective health condition events and respective health condition diagnoses; and
selecting the health-related icons comprising: selecting from the health-related icon set the at least one health condition icon representing the at least one health condition represented by the at least one health condition diagnosis.

5. The method of claim 4, generating the at least one health condition diagnosis comprising:
selecting a diagnostic weight of the respective health condition circumstance descriptors for respective health condition diagnoses from the diagnostic database;
computing an aggregate diagnostic weight for respective health condition diagnosis based on the selected diagnostic weights of the collected health condition circumstance descriptors for the health condition diagnosis; and
selecting at least one health condition diagnosis having a significant aggregate diagnostic weight.

6. The method of claim 1:
at least one health condition descriptor of the health condition record associated with at least one healthcare action descriptor representing at least one healthcare action associated with at least one health condition of the individual, comprising at least one of:
at least one healthcare test performed on the individual,
at least one healthcare procedure performed on the individual,
at least one medication prescribed for the individual, and
at least one healthcare service prescribed for the individual;
selecting the health-related icons comprising: selecting from the health-related icon set at least one healthcare action icon representing the at least one healthcare action descriptor; and
the displaying comprising: displaying the selected healthcare action icon.

7. The method of claim 6:
at least one healthcare action descriptor representing at least one healthcare action comprising at least one medication prescribed for the individual associated with at least one health condition of the individual; and
the displaying comprising: programming at least one medication instruction associated with the medication prescription into a medication device configured to store the at least one medication instruction and generate a medication reminder message by displaying icons of predominantly pictorial form from a medication regimen icon set that pertain to the medication instruction.

8. The method of claim 6:
at least one healthcare action descriptor representing at least one healthcare action comprising at least one healthcare service prescribed for the individual associated with at least one health condition of the individual; and
the displaying comprising: programming at least one service task instruction associated with the prescribed healthcare service into a service task facilitator device configured to store the at least one service task instruction and display service task icons representing the service task instructions.

9. The method of claim 1:
the health condition record of the individual comprising at least one health condition event annotation associated with at least one health condition event of the individual; and
the displaying comprising: displaying the at least one health condition event annotation with the at least one health condition event icon of the associated at least one health condition event.

10. The method of claim 1, the displaying comprising: arranging the selected icons according to at least one of:
a timeline layout representing a chronological health history of the health condition of the individual, and
a descriptive layout representing a descriptive health history of the health condition of the individual.

11. A system for representing a health condition of an individual, comprising:
a health-related icon set memory configured to store a health-related icon set comprising health-related icons of predominantly pictorial form;
a health condition record store configured to store at least one health condition record comprising:
a health condition descriptor representing the health condition of the individual, and
at least one health condition event descriptor respectively representing a health condition event associated with the health condition of the individual;
a health-related icon selecting component configured to select from the health-related icon set memory a health condition icon representing the health condition descriptor and at least one health condition event icon representing a respective health condition event descriptor; and
a displaying component configured to display the selected health condition icon and the selected at least one health condition event icon.

12. The system of claim 11:
the displaying component configured to display for a caregiver the health condition icons and health condition event icons; and
the system comprising: a health record compiling component configured to:
receive from a caregiver a selected health condition icon representing a health condition of the individual and at least one selected health condition event icon representing at least one health condition event associated with the selected health condition of the individual, and
compile a selected health condition descriptor represented by the selected health condition icon and at least one health condition event descriptor respectively represented by the selected at least one health condition event icon.

13. The system of claim 11, comprising:
a diagnostic database comprising at least one diagnostically weighted relationship between a health condition circumstance descriptor and a health diagnosis;
a diagnostic selecting component configured to select a diagnostic weight of the respective health condition circumstance descriptors for respective health condition diagnosis from the diagnostic database;
a diagnostic computing component configured to compute an aggregate diagnostic weight for respective health condition diagnosis based on the selected diagnostic weights of the health condition circumstance descriptors for the health condition diagnosis;
a diagnosing component configured to select at least one health condition diagnosis having a significant aggregate diagnostic weight; and
a health diagnosis storing component configured to store the at least one selected health condition diagnosis in the health condition record of the individual stored in the health condition record memory.

14. The system of claim 11, comprising:
at least one detection component configured to:
detect a measurement of the health condition of the individual;
select a health condition circumstance descriptor that describes the measurement from the health condition descriptor set; and
store the selected health condition circumstance descriptor in the health condition record of the individual stored in the health condition record memory.

15. The system of claim 11:
the system comprising:
at least one health condition event annotating component configured to:
receive from a caregiver at least one health condition event annotation associated with at least one health condition event of the individual, and
store the at least one health condition event annotation in the health condition record of the individual stored in the health condition record memory; and
the displaying component configured to display the at least one health condition event annotation with the at least one health condition event icon of the associated at least one health condition event.

16. The system of claim 11:
the system comprising: a medication device configured to:
store at least one medication instruction associated with at least one medication prescribed for the individual associated with at least one health condition of the individual, and
generate a medication reminder message by displaying icons of predominantly pictorial form from a medication regimen icon set that pertain to the medication instruction; and
the displaying comprising: displaying the at least one medication regimen icon with the medication reminder message associated with the at least one health condition of the individual.

17. The system of claim 11:
the system comprising: a service task facilitator device configured to:
store at least one service task instruction associated with at least one healthcare service prescribed for the individual associated with at least one health condition of the individual, and
display service task icons representing the service task instructions; and
the displaying comprising: displaying the at least one service task icon representing the at least one healthcare service associated with the at least one health condition of the individual.

18. A computer-implemented method of representing a health condition of an individual, comprising:
displaying for a caregiver a health-related icon set comprising health-related icons of predominantly pictorial form including:
at least one health condition icon representing a health condition, and
at least one health condition event icon representing a health condition event related to a health condition; and
upon receiving a selection of a health condition icon representing a health condition of the individual and at least one health condition event icon representing at least one health condition event related to the selected health condition, generating a health condition record representing the health condition of the individual and comprising:
a health condition descriptor representing the health condition associated with the selected health condition icon, and
at least one health condition event descriptor representing respective health condition events associated with the selected health condition event icons.

19. The method of claim 18, comprising:
generating at least one health condition diagnosis representing the at least one health condition of the individual based on the at least one health condition event using a diagnostic database comprising at least one correlationally weighted relationship between respective health condition events and respective health condition diagnoses; and
storing the at least one selected health condition diagnosis in the health condition record of the individual.

20. The method of claim 18, comprising:
upon receiving at least one health condition event annotation associated with at least one health condition event of the individual, storing the health condition event annotation in the health condition record.
